(19) United States
(12) Patent Application Publication
(10) Pub. No.: US 2014/0105929 A1
(43) Pub. Date: Apr. 17, 2014

(54) USE OF SARGASSUM CRISTAEFOLIUM EXTRACT

(71) Applicants: Saou-Lien Wong, Pingtung (TW); Pei-Yu Lin, Taichung City (TW)

(72) Inventors: Saou-Lien Wong, Pingtung (TW); Pei-Yu Lin, Taichung City (TW)

(73) Assignee: National Pingtung University of Science and Technology, Pingtung (TW)

(21) Appl. No.: 13/925,793

(22) Filed: Jun. 24, 2013

(30) Foreign Application Priority Data
Oct. 17, 2012 (TW) 101138283

(51) Int. Cl. A61K 36/03; A23L 1/30

(52) U.S. Cl. CPC A61K 36/03; A23L 1/30
USPC 424/195.17

(57) ABSTRACT

A use of Sargassum Cristaefolium extract in preparing a pharmaceutical or a food having a blood lipid regulation function is provided in the disclosure. The Sargassum Cristaefolium extract is obtained by performing a hot extraction process on Sargassum Cristaefolium.

### CROSS-REFERENCE TO RELATED APPLICATION

This application claims the priority benefit of Taiwan application serial no. 101138283, filed on Oct. 17, 2012. The entirety of the above-mentioned patent application is hereby incorporated by reference herein and made a part of this specification.

### BACKGROUND

1. Field of the Application

The invention relates to a use of algae extract, and more particularly, to a use of Sargassum Cristaefolium extract.

2. Description of Related Art

Lipid is one of the three major nutrients in the body and plays an essential role in maintaining a healthy physiological function; however, if lipid uptake exceeds an amount required for a physiological metabolism, excess fats would begin to accumulate, thereby causing harm to the body.

With an improvement in global economic environment, lifestyles of people gradually change, and since people tend to ingest high fat and high calorie foods, coupled with a general lack of exercise, prevalence of obesity tends to increase year by year. A study has pointed out that a high-fat diet will accelerate oxidation and inflammatory responses. Another study has noted that having an excessive cholesterol concentration will increase active oxygen substances, cause damage to endothelial cells and result in atherosclerosis, and thus is prone to develop heart and blood vascular diseases. Blood lipid abnormalities are main reasons for causing arteriosclerosis, heart diseases, cerebrovascular diseases and peripheral vascular diseases.

Currently sold hypolipidemic drugs in the market are very expensive, have many restrictions on their administration, and may contain adverse side effects. Therefore, further studies on ingredients capable of regulating blood lipid and reducing oxidative stress are in need.

Since seaweeds are rich in polysaccharides, vitamins, minerals, trace elements and so forth, they have been known as “sea vegetables”, and also have been broadly applied in a variety of food. Algae which belong to the phylum Heterokontophyta usually contain fucoidans, and the fucoidans may have characteristics of being anti-tumor and enhancing immunity. Sargassum Cristaefolium, in classification, belongs to the phylum Heterokontophyta, and the Sargassum Cristaefolium may be found throughout south and north coasts of Taiwan, and is in abundant production. However, root, stem and other portions of the Sargassum Cristaefolium are harder and poor in taste. The Sargassum Cristaefolium is not suitable for a direct consumption and is mostly only used for an undersea afforestation to provide an environmental protection or a hiding location for organisms. If such a rich ocean resource can be developed and made good use of, it definitely can bring people well-being.

### SUMMARY OF THE APPLICATION

The invention provides a use of Sargassum Cristaefolium extract, capable of improving blood lipid abnormalities in an animal body, and having an extremely low degree of damages to liver and kidney functions.

The invention provides a use of Sargassum Cristaefolium extract in preparing a pharmaceutical or a food having a blood lipid regulation function, and the Sargassum Cristaefolium extract is obtained by performing a hot extraction process on Sargassum Cristaefolium.

In an embodiment of the invention, the hot extraction process includes freeze-drying an algal body of the Sargassum Cristaefolium and grinding the algal body into powder, heating the powder in a solvent to obtain an extraction fluid, and collecting a supernatant fraction after centrifuging the extraction fluid, then freeze-drying the supernatant fraction.

In an embodiment of the invention, the solvent is distilled water.

In an embodiment of the invention, the step of heating the powder is to heat the powder in the solvent at a temperature of 93° C. to 97° C. for 4 hours to 6 hours.

In an embodiment of the invention, the extraction fluid is centrifuged at a speed of 2220 rpm.

In an embodiment of the invention, the blood lipid regulation function includes lowering a total cholesterol (TC) concentration in blood.

In an embodiment of the invention, the blood lipid regulation function includes lowering a triglyceride (TG) concentration in blood.

In an embodiment of the invention, the blood lipid regulation function includes enhancing a high-density lipoprotein cholesterol (HDL-C) concentration in blood.

In an embodiment of the invention, the blood lipid regulation function includes lowering a low-density lipoprotein cholesterol (LDL-C) concentration in blood.

In an embodiment of the invention, the blood lipid regulation function includes enhancing an antioxidant activity.

According to the foregoing, with the use of Sargassum Cristaefolium extract provided in the invention, the blood lipid abnormalities in an animal body can be improved, and the degree of damages to liver and kidney functions is extremely low.

In order to make the aforementioned and other features and advantages of the present application more comprehensible, several embodiments accompanied with figures are described in detail below.

### BRIEF DESCRIPTION OF THE DRAWINGS

The accompanying drawings are included to provide a further understanding of the application, and are incorporated in and constitute a part of this specification. The drawings illustrate embodiments of the application and, together with the description, serve to explain the principles of the application.

FIG. 1 shows total cholesterol (TC) concentration changes in blood of each experimental animal group during an experimental period according to an embodiment of the invention.

FIG. 2 shows triglyceride (TG) concentration changes in blood of each experimental animal group during an experimental period according to an embodiment of the invention.

FIG. 3 shows high-density lipoprotein (HDL-C) concentration changes in blood of each experimental animal group during an experimental period according to an embodiment of the invention.

FIG. 4 shows low-density lipoprotein cholesterol (LDL-C) concentration changes in blood of each experimental animal group during an experimental period according to an embodiment of the invention.

### DETAILED DESCRIPTION OF DISCLOSED EMBODIMENTS

The invention provides a use of Sargassum Cristaefolium extract in preparing a pharmaceutical or a food having a blood lipid regulation function, and the Sargassum Cristaefolium extract is obtained by performing a hot extraction process on Sargassum Cristaefolium.

The hot extraction process may include the following steps (I) to (III).

Step (I): freeze-drying an algal body of the Sargassum Cristaefolium, and the algal body is ground into powder. The Sargassum Cristaefolium, for example, is collected from Wanlitong area in Kenting, Taiwan, but not limited thereto, and the algal body refers to the entire Sargassum Cristaefolium (including root, stem and leaf portions). Since topography of the Wanlitong area in Kenting is suitable for collecting algae and the algal bodies in this area are usually in a healthy condition, the Sargassum Cristaefolium in the following experiments is collected from there. A rich harvest period of the Sargassum Cristaefolium in this area is annually from March till May.

Step (II): heating the powder in a solvent to obtain an extraction fluid. In this step, the solvent, for example, is distilled water; however, other solvents not affecting the effect of the extract in lowering the blood lipid may also be considered; and the powder, for example, is heated in the solvent at a temperature of 93° C. to 97° C. for 4 hours to 6 hours, but substantially not limited thereto. The aforementioned heating method, for example, is to perform a hot water extraction under 95° C. using a hot bath machine for 5 hours.

Step (III): centrifuging the extraction fluid, then a supernatant fraction is collected and is freeze-dried. The centrifugation of the extraction fluid, for example, is performed at a speed of 2220 rpm, and a centrifugation time, for example, is 10 minutes, but those skilled in the art should be able to understand that the requirements for performing the centrifugation are not specifically limited thereto, as long as the extraction fluid may be stratified.

In addition, the blood lipid regulation function may include lowering a total cholesterol (TC) concentration in the blood, lowering a triglyceride (TG) concentration in the blood, enhancing a high-density lipoprotein cholesterol (HDL-C) concentration in the blood, lowering a low-density lipoprotein cholesterol (LDL-C) concentration in the blood and enhancing an antioxidant activity, but not limited thereto.

In the following paragraphs, experimental examples are presented to further explain the materials and the extraction method of the Sargassum Cristaefolium extract provided in the invention, and it is to be noted that, actual implementations of the invention are not limited thereto, those skilled in the art should be able to deduce the other implementations of the invention based on the disclosure.

Sargassum Cristaefolium Acquisition and Hot Extraction Process

The Sargassum Cristaefolium in the present experimental example was collected from Wanlitong area, Hengchun, Taiwan, during a low tide period in February, 2010. The collected algal body was immediately being placed at a low temperature condition (about 4° C.). Then, after the algal body was rinsed with fresh water to remove impure algae, freeze-drying was performed, the algal body was ground into powder, and 4 g of powder was added into 100 mL of distilled water for soaking and being heated in the distilled water at 95° C. for 5 hours. Afterward, the extraction fluid was disposed in a centrifuge tube under 4° C. and was centrifuged at the speed of 2220 rpm for 15 minutes, and the supernatant obtained from the resulting extraction fluid, after underwent a freeze-drying process performed by a freeze-drying machine (Eyela, FDU-1100, Tokyo, Japan), was stored at −20° C. for future use.

Component Analysis of Sargassum Cristaefolium Extract

A. Analysis of General Components

After a general component analysis was performed on the Sargassum Cristaefolium extract (powder), the result showed that, a crude protein content thereof was 3.7±0.1%, a crude lipid content thereof was 2.0±0.7%, a crude fiber content thereof was 17.8±0.8%, and a crude ash content thereof was 25.6±0.6% (all values are expressed in weight percentage).

B. Analysis of Antioxidants

An analysis of antioxidants was performed on the Sargassum Cristaefolium extract, and the analysis result showed that, a polyphenol content thereof was 27.7±6.6 μg/ml (μg gallic acid equivalent/ml), a polysaccharide content thereof was 18.2±0.05%, a flavonoid content thereof was 11.2±1.5 μg/ml (μg quercetin/ml), a chlorophyll content thereof was 25.8±0.4 μg/g, and a carotenoid content thereof was 6.1±3.0 μg/g.

In addition, an antioxidant capacity analysis showed that, a reducing power thereof was 49.4±8.0%, a superoxide dismutase (SOD) content thereof was 31.3±4.1%, DPPH (2,2-diphenyl-1-picrylhydrazyl) scavenging capability thereof was 47.4±2.1%, and a ferrous ion chelating activity thereof was 13.8±2.5%. The abovementioned “%” is expressed in weight percentage. The polyphenol content and the flavonoid content were acquired by conducting a test with the extract (powder) aqueous solution of 100% concentration.

Accordingly, the Sargassum Cristaefolium extract is rich in antioxidants, such as polyphenolic compounds, and the ferrous ion chelating activity, the DPPH scavenging capability and the reducing power thereof are also excellent. Therefore, after ingested by an animal body, an antioxidant activity within in the animal body can be enhanced, a lipid peroxidation can be lowered, and inflammatory responses may be alleviated.

Application Evaluation of Sargassum Cristaefolium Extract in Blood lipid Regulation Function

A. Experimental Design

In the present experimental example, hamsters with a cholesterol and bile acid metabolism much more similar to that of human were being used as animal models. The hamsters beings used in the present experimental example were 50 Golden Syrian hamsters (about seven weeks old) bought from the National Experimental Animal Center (Nan-Kang, Taipei, Taiwan). The 50 hamsters were randomly divided into five groups, which respectively were: a control group (hereinafter sometimes abbreviated as “CL”), a high fat group (hereinafter sometimes abbreviated as “HF”), a 0.8% Sargassum Cristaefolium extract-added group (hereinafter sometimes abbreviated as “HF+0.8 S”), a 1.6% Sargassum Cristaefolium extract-added group (hereinafter sometimes abbreviated as “HF+1.6 S”), and a 2.4% Sargassum Cristaefolium extract-added group (hereinafter sometimes abbreviated as “HF+2.4 S”).

Firstly, except the control group (CL), each group was fed with a high fat and high cholesterol feed for four weeks so as to induce hyperlipidemia (TG: not less than 200 mg/dl; TC: not less than 200 mg/dl).

The feed described above uses Laboratory rodent feeding diet 5001 (YoungLi) as its basal feed. In addition, the high fat and the cholesterol feed were prepared by further adding 0.2% cholesterol, 10% lard and a proper amount of distilled water in the basal feed.

After feeding the groups (except the control group) with the high fat and cholesterol feed for four weeks, a ten week experimental period was then started. Components of the feed fed to each group of hamsters in the experimental period are listed in Table 1 below.

TABLE 1

| | | | | HF + | HF + |
|---|---|---|---|---|---|
| | CL | HF | HF + 0.8S | 1.6S | 2.4S |
| Laboratory rodent diet 5001 | 100 | 89.8 | 89 | 88.2 | 87.4 |
| Lard | 0 | 10 | 10 | 10 | 10 |
| Cholesterol | 0 | 0.2 | 0.2 | 0.2 | 0.2 |
| Sargassum Cristaefolium extract | 0 | 0 | 0.8 | 1.6 | 2.4 |

Unit: weight percentage (%)

In addition, the feed containing the Sargassum Cristaefolium extract were prepared by adding the Sargassum Cristaefolium extract of different weights into the basal feed according to the groups; after being homogeneously stirred, the feed was extruded into cylindrical bars by a meat grinder (with an aperture of 1.2 cm) and was placed on plates, then the plates were placed into an oven for drying at a low temperature of 50° C.; after cooling down, the feed was stored in a refrigerator for ensuring composition stability.

Weights and feeding volumes of the hamsters were recorded each week, and growth conditions of the hamsters were also observed. In addition, before entering the ten week experimental period, an orbital venous plexus blood collection (1 mL, and 24 hours of fasting before the blood collection) was performed on the hamsters, and blood biochemical analysis values acquired at the time were to be used as basic values. After entered the ten week experimental period, blood collections were performed at 2^{nd}, 4^{th}, 6^{th}, 8^{th} and 10^{th} week for verifying the changes in the blood lipid.

After the experiment ended, the experimental animals were pathologically dissected, and hearts, livers, pancreas, kidneys and adipose tissues thereof were collected for performing lipid analysis and tissue slices.

B. Blood Biochemical Analysis Method

A determination of the total cholesterol concentration (TC) was carried out by using a Fortress cholesterol reagent (Diagnostics Cholesterol, CHOD-PAP), and a determination of the triglyceride concentration (TG) in the blood was carried out by using Fortress triglyceride reagent (GPO-PAP). The determination method was to add 10 μL of a serum sample into 1000 μL of reagent, left to stand at a temperature of 20° C. to 25° C. for 10 minutes, wait until the serum sample shows a color reaction, use a spectrophotometer to determine a light absorption value of the serum sample at a wavelength of 500 nm, and then compare the light absorption value with a light absorption value of a standard solution to obtain the TC and TG concentrations of the serum sample. The abovementioned serum sample is a supernatant obtained by centrifuging the blood of the hamsters at 3000 rpm for 10 minutes.

Determination of the high-density lipoprotein cholesterol (HDL-C) and the low-density lipoprotein cholesterol (LDL-C) in the blood adopted an enzymatic reaction and a CHOP-PAP method. A quantitative amount of the serum sample was added into a precipitating agent (which contains phosphotungstic acid and magnesium chloride), and as highand low-density lipoproteins are respectively separated, the HDL-C and LDL-C in the blood were determined using the aforementioned total cholesterol determination method. Namely, the HDL-C and LDL-C concentrations in the blood were acquired by determining the contents of HDL-C and LDL-C in the plasma.

Experimental result data were all expressed in mean±stand deviation (SD). In the data analysis, differences between the groups were compared by using a SAS system (statistical analysis system) to perform a single-factor analysis on variance (ANOVA) and then by using a Duncan's multiple range test, and p<0.05 was regarded as having significant differences.

C. Component Analysis of Liver and Feces

Liver lipid extraction: 0.5 g of liver sample was added into a Folch solution (chloroform:methanol=2:1; v/v) equal to 20 times the liver sample weight, the liver cells were ground with a grinding rod, 10 μL of liver lipid extraction fluid was added into 10 μL Triton X-100, the resulting solution was mixed evenly and blow dried, and then the aforementioned TC and TG reagents were used to analyze the cholesterol and triglyceride contents in the liver.

An analysis of the feces lipid was performed by adding a quantitative amount of feces powder into a Folch solution (chloroform:methanol=2:1; v/v) equal to 20 times the feces powder weight, after being homogenized, the resulting solution was shock extracted for 12 hours at the room temperature and filtered with a filter paper, the feces sample was then quantified to 10 mL via the Folch extraction fluid. Similarly, the cholesterol and triglyceride contents in the liver were analyzed using the aforementioned TC and TG reagents.

An analysis of neutral steroids used a modified method based on a method established by Folch (1957) et al. 0.1 mL of extraction fluid was placed in a fume hood for volatilization, and then 1 mL of Liberman Burchard Reagent (acetic anhydride:sulfuric acid:acetic acid) was added; after being evenly mixed and reacted under the room temperature for 20 minutes, a spectrophotometer was used to measure the light absorption value (at a wavelength of 550 nm) of the extraction fluid, and the neutral steroid content (%) of the feces was calculated according to a standard curve established by using a cholesterol standard (200 mg/dl) of the cholesterol reagent.

An analysis of bile acid was to add 0.3 g of feces powder into 2 mL of 95% methanol to perform an extraction for 5 minutes in an ice bath; next, use the centrifuge machine to centrifuge the resulting sample solution at 3000 rpm for 10 minutes; after repeating the above steps twice, use 245 mL of petroleum ether to extract a supernatant of the sample solution; after condensing the supernatant, use the methanol to quantify the sample solution to 1 ml; use a bile acid reagent of Fortress to perform measurements.

D. Tissue Slice Production

After dissecting the hamsters, hearts, livers, kidneys and lesion locations thereof were soaked in 10% neutral formalin; after 18 to 24 hours of fixation, steps of dehydration, paraffin embedding, slicing and so forth were performed; finally, after H&E (Hematoxylin and Eosin) dyeing was performed, tissue slices were observed and recorded under an optical microscope.

E. Experimental Results

During the experimental period, there was no fur color difference between each group of animals and no depilation observed, and activity conditions and reactions of the animals were all normal. No significant difference was observed in initial body weights and final body weights between each group. In addition, also no significant difference was observed in initial feed intake and final feed intake between each group.

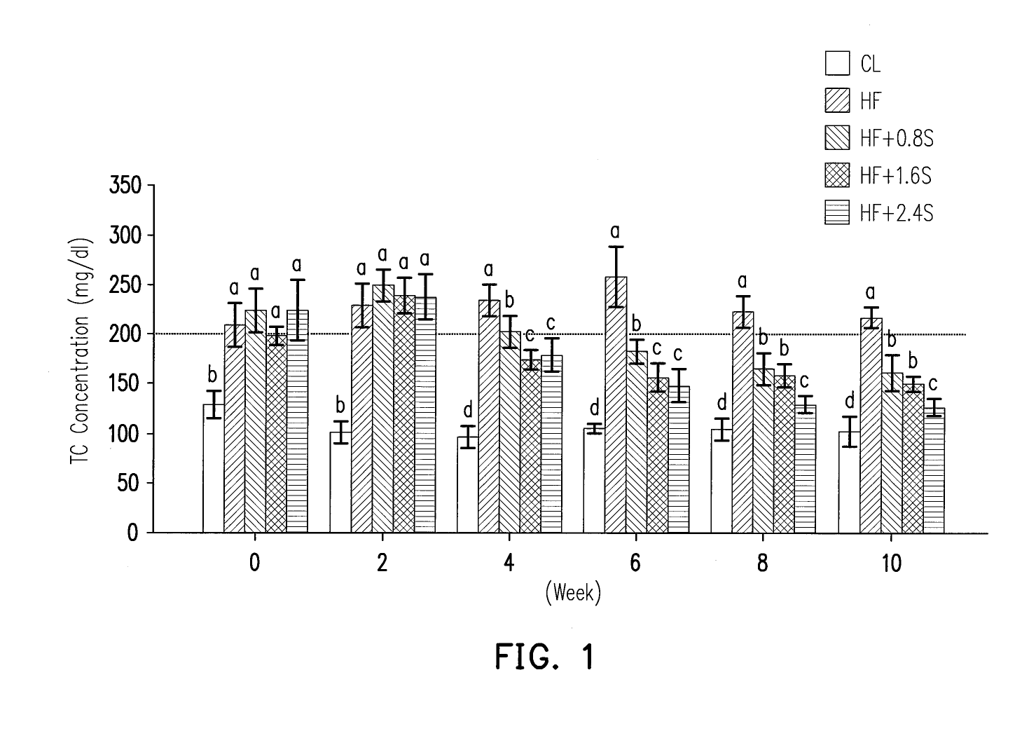

The aforementioned blood biochemical analysis results of the hamsters are shown in FIG. 1 to FIG. 4. The data in each figure are all expressed in mean±SD (n=10). Data having different superscript letters in the same row represent that the difference among them is significant (p<0.05), and these data are results acquired according to Duncan's multiple range test.

Total Cholesterol (TC) Concentration Change in Blood

After being fed with the high fat and high cholesterol feed for four weeks, the total cholesterol concentration in the blood of the experimental animals was 213.8±23.68 mg/dl, and was significantly higher than that of the control group (CL)(p<0.05). Using abovementioned value as an initial value, results of the total cholesterol concentration changes in the blood of each group of experimental animals after entering the ten week experimental period are shown in FIG. 1.

Referring to FIG. 1, the total cholesterol concentration in the blood of the control group (CL) fed only with the basal feed is significantly lower than that of the other groups during the experimental period. In addition, during the experimental period, the total cholesterol concentration of the 0.8% extract-added group (HF+0.8 S) decreases from 223.7±20.9 mg/dl to 162.5±17.9 mg/dl, which is obviously reduced by 27.4%; the total cholesterol concentration of the 1.6% extract-added group (HF+1.6 S) decreases from 198.66±8.4 mg/dl to 151.0±7.2 mg/dl, which is obviously reduced by 24%; the total cholesterol concentration of the 2.4% extract-added group (HF+2.4 S) decreases from 224.2±16.2 mg/dl to 127.3±7.6 mg/dl, which is obviously reduced by 43.2%.

It may be known from the above experimental results that, as the additive amount of the extract increases, the descent rate of the total cholesterol concentration in the blood also increases, and increase of the descent rate is time-dependent.

Triglyceride (TG) Concentration Change in Blood

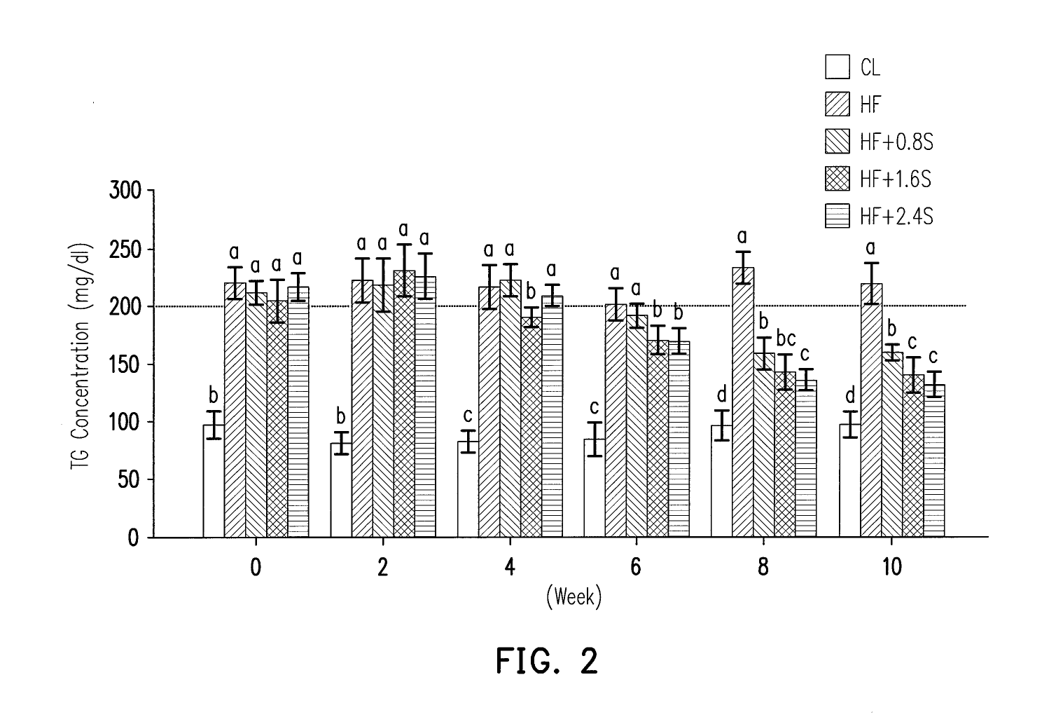

After being fed with the high fat and high cholesterol feed for four weeks, the triglyceride concentration in the blood of the experimental animals was 214.04±6.6 mg/dl, which is significantly higher than that of the control group (CL)(p<0.05). Using abovementioned value as an initial value, results of the triglyceride concentration changes in the blood of each group of experimental animals after entering the ten week experimental period are shown in FIG. 2.

Referring to FIG. 2, the triglyceride concentration in the blood of the control group (CL) fed only with the basal feed is significantly lower than that of the other groups during the experimental period. In addition, during the experimental period, the triglyceride concentration of the 0.8% extract-added group (HF+0.8 S) decreases from 212.5±9.7 mg/dl to 161.8±5.8 mg/dl, which is obviously reduced by 23.9%; the triglyceride concentration of the 1.6% extract-added group (HF+1.6 S) decreases from 205.6±18.4 mg/dl to 142.1±14.9 mg/dl, which is obviously reduced by 31%; the triglyceride concentration of the 2.4% extract-added group (HF+2.4 S) decreases from 217.1±12.3 mg/dl to 134.1±10.6 mg/dl, which is obviously reduced by 38.2%.

It may be known from the above experimental results that, as the additive amount of the extract increases, the descent rate of the triglyceride concentration in the blood also increases, and increase of the descent rate is time-dependent.

High-Density Lipoprotein Cholesterol (HDL-C) Concentration Change in Blood

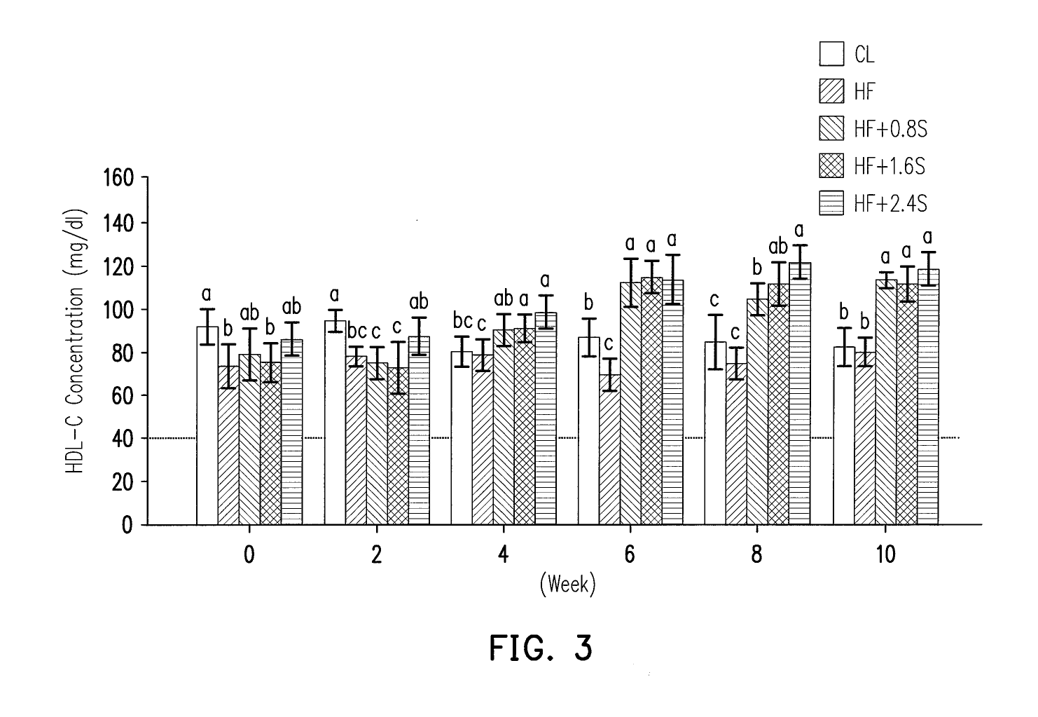

FIG. 3 shows high-density lipoprotein (HDL-C) concentration changes in blood of each experimental animal group during an experimental period according to an embodiment of the invention.

After being fed with the high fat and high cholesterol feed for four weeks, the HDL-C concentration in the blood of the experimental animals had become lower. Results of the HDL-C concentration changes in the blood of each group of experimental animals after entering the ten week experimental period are shown in FIG. 3.

Referring to FIG. 3, the HDL-C concentration in the blood of the control group (CL) fed only with the basal feed is significantly lower than that of the other groups during the experimental period. In addition, during the experimental period, the HDL-C concentration of the 0.8% extract-added group (HF+0.8 S) increases from 79.2±11.6 mg/dl to 114.1±3.7 mg/dl, which is obviously increased by 44.1%; the HDL-C concentration of the 1.6% extract-added group (HF+1.6 S) increases from 75.3±8.6 mg/dl to 112.2±8.3 mg/dl, which is obviously increased by 49%; the HDL-C concentration of the 2.4% extract-added group (HF+2.4 S) increases from 85.9±7.9 mg/dl to 118.9±8.4 mg/dl, which is obviously increased by 38.4%.

It may be known from the above experimental results that, the HDL-C concentration in the blood of each group of hamsters fed with the feed containing the Sargassum Cristaefolium extract is significantly higher than that of the control group (CL) and the high fat group (HF) (6^{th}, 8^{th}, 10^{th} weeks), and this shows that the Sargassum Cristaefolium extract indeed can enhance the HDL-C concentration in the blood. The higher the HDL-C concentration in the blood, the easier it is for the cholesterols in the blood to be carried to and stored in the liver, and thus may assist in lowering the cholesterol concentration in the blood, thereby reducing a risk of having arteriosclerosis.

Low-Density Lipoprotein Cholesterol (LDL-C) Concentration Change in Blood

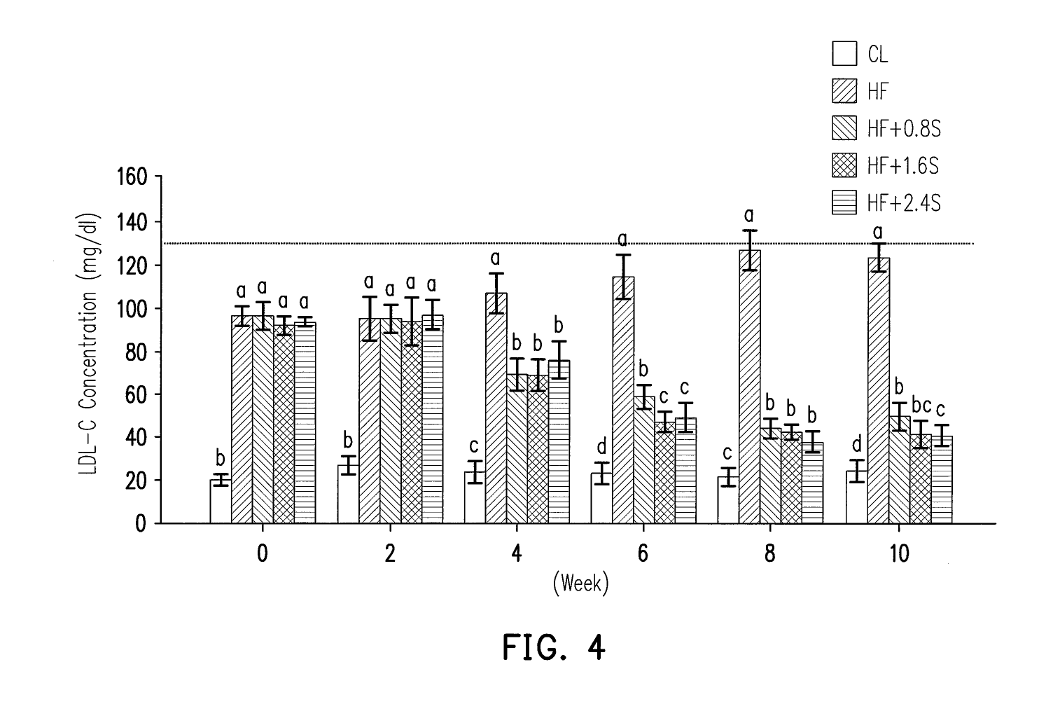

FIG. 4 shows low-density lipoprotein cholesterol (LDL-C) concentration changes in blood of each experimental animal group during an experimental period according to an embodiment of the invention.

After being fed with the high fat and high cholesterol feed for four weeks, the LDL-C concentration in the blood of the experimental animals is significantly higher than that of the control group (CL)(p<0.05). Results of the LDL-C concentration changes in the blood of each group of experimental animals after entering the ten week experimental period are shown in FIG. 4.

Referring to FIG. 4, the LDL-C concentration in the blood of the control group (CL) fed only with the basal feed is significantly higher than that of the other groups during the experimental period. In addition, during the experimental period, the LDL-C concentration of the 0.8% extract-added group (HF+0.8 S) decreases from 96.4±6.5 mg/dl to 49.7±6.8 mg/dl, which is obviously reduced by 48.4%; the LDL-C concentration of the 1.6% extract-added group (HF+1.6 S) decreases from 91.9±4.6 mg/dl to 41.3±6.6 mg/dl, which is obviously reduced by 55%; the LDL-C concentration of the 2.4% extract-added group (HF+2.4 S) decreases from 93.4±2.5 mg/dl to 40.3±5.2 mg/dl, which is obviously reduced by 56.9%.

It may be known from the above experimental results that, during the 4^{th} to the 10^{th} weeks of the experimental period, the LDL-C concentration in the blood of each group of hamsters fed with the feed containing the Sargassum Cristaefolium extract is significantly lower than that of the high fat group (HF), and this is presumably because that the Sargassum Cristaefolium extract can speed up the metabolism of triglycerides, thus slowing down the generation of LDL-C in the liver.

Changes of LDL-C/HDL-C and HDL-C/TC Ratios

As generally known, in order to assist in regulating blood lipid levels, at least one of the following two requirements has to be met: (1) a proportion of LDL-C/HDL-C ratio is reduced or a proportion of HDL-C/TC is increased; (2) the proportion of the LDL-C/HDL-C remains unchanged, but the proportion of HDL-C/TC obviously decreases.

In the present study, each group of experimental animals, after being fed with the high fat feed for four weeks, changes of the LDL-C/HDL-C ratio in the blood thereof are as shown in Table 2.

TABLE 2

| | Week | | | | | |
|---|---|---|---|---|---|---|
| Group | 0 | 2 | 4 | 6 | 8 | 10 |
| Control | 0.21 ± 0.03^{c} | 0.28 ± 0.05^{b} | 0.29 ± 0.07^{c} | 0.27 ± 0.06^{c} | 0.26 ± 0.07^{c} | 0.31 ± 0.13^{b} |
| group | | | | | | |
| (CL) | | | | | | |
| High fat | 0.65 ± 0.15^{a} | 1.22 ± 0.14^{a} | 1.37 ± 0.16^{a} | 1.66 ± 0.22^{a} | 1.71 ± 0.20^{a} | 1.55 ± 0.13^{a} |
| group | | | | | | |
| (HF) | | | | | | |
| HF + 0.8S | 0.59 ± 0.08^{ab} | 1.28 ± 0.18^{a} | 0.77 ± 0.11^{b} | 0.53 ± 0.07^{b} | 0.42 ± 0.04^{b} | 0.44 ± 0.06^{b} |
| HF + 1.6S | 0.56 ± 0.10^{ab} | 1.33 ± 0.32^{a} | 0.76 ± 0.12^{b} | 0.41 ± 0.05^{bc} | 0.38 ± 0.06^{bc} | 0.37 ± 0.07^{b} |
| HF + 2.4S | 0.51 ± 0.06^{b} | 1.12 ± 0.16^{a} | 0.77 ± 0.11^{b} | 0.43 + 0.07^{b} | 0.31 ± 0.04^{bc} | 0.34 ± 0.04^{b} |

Data are all expressed in mean ± SD (n = 10). Data having different superscript letters in the same row represents that the difference among them is significant (p < 0.05), and these data are results acquired according to Duncan's multiple range test.

As shown in Table 2, the LDL-C/HDL-C ratios of the induced experimental animals are significantly higher than that of the control group (p<0.05). When the experimental period enters the 4^{th} week, it can be found that the LDL-C/HDL-C ratio in the blood of each group of hamsters fed with the Sargassum Cristaefolium extract is obviously lower than that of the high fat group; until the end of the experimental period, the LDL-C/HDL-C ratio in the blood of each group of hamsters fed with the Sargassum Cristaefolium extract is lower than that of the high fat group; the higher the ratios are, the higher probabilities of developing cardiovascular diseases. It is speculated that the Sargassum Cristaefolium extract reduces the LDL-C/HDL-C ratio by reducing the LDL-C content in the blood.

Having a higher ratio of HDL-C/TC has a better effect of preventing or ameliorating the cardiovascular diseases. After being fed with the high fat feed for four weeks, the changes of the HDL-C/TC ratio in the blood in each group of experimental animals are as shown in Table 3.

TABLE 3

| | Week | | | | | |
|---|---|---|---|---|---|---|
| Group | 0 | 2 | 4 | 6 | 8 | 10 |
| Control | 0.72 ± 0.09^{a} | 0.93 ± 0.10^{a} | 0.84 ± 0.11^{a} | 0.83 ± 0.08^{a} | 0.82 ± 0.15^{ab} | 0.81 ± 0.16^{ab} |
| group | | | | | | |
| (CL) | | | | | | |
| High fat | 0.35 ± 0.06^{b} | 0.34 ± 0.03^{b} | 0.34 ± 0.04d | 0.27 ± 0.03^{c} | 0.34 ± 0.03d | 0.37 ± 0.05^{c} |
| group | | | | | | |
| (HF) | | | | | | |
| HF + 0.8S | 0.36 ± 0.07^{b} | 0.30 ± 0.03^{b} | 0.45 ± 0.05^{c} | 0.62 ± 0.09^{a} | 0.64 ± 0.07^{c} | 0.71 ± 0.09^{b} |
| HF + 1.6S | 0.38 ± 0.05^{b} | 0.31 ± 0.06^{b} | 0.52 ± 0.05^{bc} | 0.74 ± 0.07^{b} | 0.71 ± 0.11^{bc} | 0.75 ± 0.08^{b} |
| HF + 2.4S | 0.39 ± 0.05^{b} | 0.37 ± 0.07^{b} | 0.55 ± 0.06^{b} | 0.78 ± 0.16^{a} | 0.95 ± 0.09^{a} | 0.94 ± 0.09^{a} |

Data are all expressed in mean ± SD (n = 10). Data having different superscript letters in the same row represents that the difference among them is significant (p < 0.05), and these data are results acquired according to Duncan's multiple range test.

As shown in Table 3, the HDL-C/TC ratios of the induced experimental animals are significantly lower than that of the control group (p<0.05). When the experimental period enters the 4^{th} week, it can be found that the HDL-C/TC ratio in the blood of each group of hamsters fed with the Sargassum Cristaefolium extract is significantly higher than that of the high fat group; until the end of the experimental period, the HDL-C/TC ratio in the blood of each group of hamsters fed with the Sargassum Cristaefolium extract is higher than that of the high fat group; the higher the ratios are, the lower probabilities of developing cardiovascular diseases. As shown by the experimental results, it is speculated that the Sargassum Cristaefolium extract increases the HDL-C/TC ratio by increasing the HDL-C content in the blood.

Safety Assessment of Sargassum Cristaefolium Extract

Glutamate oxaloacetate transaminase (GOT) and glutamyl pyrubic transaminase (GPT) are important enzymes within liver cells, when the liver cells are suffering damages, these types of enzymes are released into the blood. Therefore, concentrations of these enzymes are often being detected, medically, as a method for evaluating a degree of damage of the liver cells. A concentration of blood urea nitrogen (BUN) is also a common clinically used kidney function index. If the concentration of blood urea nitrogen is too high, it represents that the kidney is not able to successfully excrete the urea nitrogen. Therefore, the concentration of blood urea nitrogen may be used for evaluating kidney diseases.

In the present experiment, influences of feeding the Sargassum Cristaefolium extract on the livers and the kidneys of the experimental animals are also evaluated by detecting GOT, GPT and BUN levels in the blood of the experimental animals. Results are shown below in Table 4. After the 10 weeks of experimental period, there is no significant difference among the extract-added group, the high fat group (HF) and the control group (CL) of the experimental animals. This shows that feeding the Sargassum Cristaefolium extract does not influence the liver and kidney functions of the hamsters.

TABLE 4

| | CL | HF | HF + 0.8S | HF + 1.6S | HF + 2.4S |
|---|---|---|---|---|---|
| GOT (U/g) | 84.33 ± 6.35 | 91.50 ± 4.09 | 90.83 ± 5.34 | 86.67 ± 6.56 | 83.83 ± 4.07 |
| GPT (U/g) | 79.67 ± 9.03 | 89.83 ± 7.03 | 80.17 ± 3.71 | 82.00 ± 4.65 | 79.17 ± 7.60 |
| BUN (mg/dl) | 23.12 ± 2.97 | 21.78 ± 2.39 | 22.23 ± 2.24 | 22.07 ± 2.32 | 22.67 ± 2.64 |

Data are all expressed in mean ± SD (n = 10).
Data having different superscript letters in the same row represents that the difference among them is significant (p < 0.05), and these data are results acquired according to Duncan's multiple range test.

Component Analysis of Liver Lipid

As shown in Table 5 below, during the experimental period, the total cholesterol content in the liver of the high fat group (HF) is significantly higher than that of the control group (CL). The total cholesterol contents in the livers of the other groups are all significantly lower than that of the high fat group. Among these groups, the 2.4% extract-added group (HF+2.4 S) has the lowest total cholesterol content. It is speculated to be a result of a large amount of water-soluble dietary fibers contained in the Sargassum Cristaefolium extract.

In addition, the triglyceride content in the liver of the high fat group (HF) liver is also significantly higher than that of the control group (CL), and it shows that a high fat diet may cause accumulations of the total cholesterol and the triglyceride. The triglyceride contents in the livers of the other groups are all significantly lower than that of the high fat group. Comparing the results of each group, it is found that the triglyceride content tends to reduce as the Sargassum Cristaefolium extract increases. It can be known from above that the Sargassum Cristaefolium extract can influence the lipid metabolism in the liver and reduce the triglyceride content in the liver.

TABLE 5

| | CL | HF | HF + 0.8S | HF + 1.6S | HF + 2.4S |
|---|---|---|---|---|---|
| TC | 27.45 ± 2.56^{c} | 56.70 ± 2.91^{a} | 44.09 ± 1.82^{b} | 43.36 ± 1.73^{b} | 42.39 ± 1.47^{b} |
| (mg/dl) | | | | | |
| TG | 34.90 ± 1.85^{c} | 63.20 ± 2.25^{a} | 45.16 ± 2.68^{b} | 42.52 ± 2.78^{b} | 40.87 ± 1.09^{bc} |
| (mg/dl) | | | | | |

Data are all expressed in mean ± SD (n = 10).
Data having different superscript letters in the same row represents that the difference among them is significant (p < 0.05), and these data are results acquired according to Duncan's multiple range test.

Component Analysis of Feces Lipid

As shown in Table 6 below, during the experimental period, there is no significant difference between the total cholesterol content in the feces of the high fat group (HF) and that of the control group (CL), and there is also no significant difference between the other groups. It shows that the mechanism of the Sargassum Cristaefolium extract for reducing cholesterols in the serum is not by increasing a cholesterol excretion in the feces. The triglyceride content in the feces of the high fat group (HF) is significantly higher than that of the control group (CL); whereas in the other groups, the triglyceride contents in the feces are all higher than that of the high fat group (HF). It is speculated that addition of the Sargassum Cristaefolium extract would not cause triglycerides to accumulate in the liver.

As described above, the triglyceride content in the feces of the high fat group (HF) is obviously higher than that of the control group (CL), but there is has no significant difference between the cholesterol content of the high fat group (HF) and that of the control group (CL). It may be deduced that a mechanism inside the body can store excess cholesterol in the liver, thereby causing the cholesterol content in the feces to decrease.

In addition, the neutral steroid content in the feces of the high fat group (HF) is significantly lower than that of the control group (CL), and the neutral steroid content of each extract-added group is significantly higher than the high fat group (HF) content. Therefore, it is speculated that adding the Sargassum Cristaefolium extract would increase the neutral steroid content in the feces, thereby achieving an effect of reducing blood lipids. There is no significant difference between the bile acid content in the feces of the high fat group (HF) and that of the control group, and the bile acid content of each extract-added group is significantly higher than the high fat group content. It may be seem from the above results that adding the Sargassum Cristaefolium extract may also increase an excretion of feces bile acid.

TABLE 6

| | CL | HF | HF + 0.8S | HF + 1.6S | HF + 2.4S |
|---|---|---|---|---|---|
| TC (mg/dl) | 1.48 ± 0.04 | 1.46 ± 0.04 | 1.52 ± 0.02 | 1.48 ± 0.05 | 1.51 ± 0.03 |
| TG (mg/dl) | 1.56 ± 0.02^{c} | 1.87 ± 0.04^{b} | 1.87 ± 0.02^{b} | 1.95 ± 0.02^{a} | 1.97 ± 0.04^{a} |
| Neutral steroid | 8.97 ± 0.25^{b} | 6.27 ± 1.15^{c} | 16.27 ± 0.95^{a} | 11.43 ± 0.99^{b} | 9.40 ± 0.40^{b} |
| (mg/g) | | | | | |
| Feces bile acid | 4.1 ± 0.10^{c} | 4.87 ± 0.15^{bc} | 5.43 ± 0.42^{ab} | 5.47 ± 0.40^{ab} | 6.07 ± 0.21^{a} |
| (mg/g) | | | | | |

Data are all expressed in mean ± SD (n = 10).
Data having different superscript letters in the same row represents that the difference among them is significant (p < 0.05), and these data are results acquired according to Duncan's multiple range test.

Results of Tissue Slice Analysis

The control group (CL) had no histopathological change in heart tissues and coronary arteries. The heart tissues of the high fat group (HF) had a condition of atherosclerosis, and myocardial fibers became irregular wavy-shaped arrangements. That is mainly due to long-term ingestion of high fat diet, such that fat accumulates and obstruct coronary arteries, and unstable plaques are formed. All of the hearts and the coronary arteries of the rest of the groups had no tissue pathological change.

The liver tissue slices of the control group (CL) were normal and without histopathological change, while the liver tissue slices of the high fat group (HF) had fat accumulations and cell swellings at partial regions around the liver. Each extract-added group only showed a slight histopathological change.

The kidney tissue slices of each group had no histopathological change and no symptom of inflammation. It is speculated that addition of the Sargassum Cristaefolium extract may not cause pathological changes in the kidneys of the hamsters. In addition, the kidneys of the control group did not have any pathological changes. While regions around the kidneys of the high fat group and the HF+0.8% S group were obviously covered by fat cells, the kidneys of the HF+1.6% S group and the HF+2.4% S group had no significant abnormality. The results showed that addition of the Sargassum Cristaefolium extract can significantly suppress an increase of fat cells around the kidney and alleviate a condition of adipose tissue accumulation. The possible reasons may include enhancing an efficiency in decomposing fats in the adipose tissues around the kidney.

The experimental results showed that the ingestion of the Sargassum Cristaefolium extract may reduce the concentrations of triglyceride (TG), cholesterol (TC) and low-density lipoprotein cholesterol (LDL-C) in the blood while enhancing the concentration of high-density lipoprotein cholesterol (HDL-C). As for the mechanism of the Sargassum Cristaefolium extract in reducing the blood lipid, it is speculated that, by increasing the excretions of neutral cholesterols and bile acids in feces, the liver is stimulated to metabolize cholesterols to bile acids, thereby achieving the effect of reducing the blood lipid.

In addition, since the Sargassum Cristaefolium extract is rich in antioxidants, it may slow down a damage in liver which is caused by lipid peroxidation, and may lower an occurrence of acute swelling and the necrotizing hepatitis in the liver, such that the Sargassum Cristaefolium extract is quite suitable for preparing a food or a pharmaceutical having a blood lipid regulation function.

It is speculated that, since the Sargassum Cristaefolium contains a large amount of fucoidans, it has characteristics of being anti-tumor, enhancing immune activity, being nontoxic and so forth. Therefore, the Sargassum Cristaefolium may be applied to applications of antibacterial, anti-tumor, blood cleaning, cholesterol regulation, blood pressure reduction, cervical lymphadenopathy treatment, edema elimination, anti-inflammatory, antipyretic, diuretic, human colon cancer cell proliferation inhibition and so forth.

In the aforementioned experiment, the Sargassum Cristaefolium extract was directly mixed into food, and the additive amount thereof was approximately calculated with reference to a serving suggestion of an commercially available algae extract product, wherein a recommended dose for an average adult is about 4 g per day. By calculating with a total food intake of 500 g (dry weight) per person per day, the recommended dose of the Sargassum Cristaefolium is about 0.8% of the total food intake. Therefore, the aforementioned feed was designed by making an addition of 0.8% extract as a basic additive amount. Specifically, when the Sargassum Cristaefolium extract of the invention is practically used, a usage amount thereof can be referred to a health food intake suggestion proposed by the Examination Department of Health. Namely, for an average adult, the daily Sargassum Cristaefolium extract intake can be approximately 0.8% of the total food intake.

However, those skilled in the art should be able to know that no matter it is to apply the Sargassum Cristaefolium extract in preparing the food or the pharmaceutical having the blood lipid regulation function, the usage amount and the intake thereof should be determined by actual needs and individual differences.

In summary, with the applications of the Sargassum Cristaefolium extract provided by the invention, the blood lipid abnormalities in an animal body can be improved, and a degree of damages to liver and kidney functions liver is extremely low. For people prone to high risks of developing cardiovascular diseases or patients easily experience allergic reactions to hypolipidemic drugs, the Sargassum Cristaefolium extract may also be classified as a reference of hypolipidemic substance.

It will be apparent to those skilled in the art that various modifications and variations can be made to the structure of the application without departing from the scope or spirit of the application. In view of the foregoing, it is intended that the application cover modifications and variations of this application provided they fall within the scope of the following claims and their equivalents.

What is claimed is:

1. A use of Sargassum Cristaefolium extract in preparing a pharmaceutical or a food having a blood lipid regulation function, wherein the Sargassum Cristaefolium extract is obtained by performing a hot extraction process on Sargassum Cristaefolium.

2. The use of Sargassum Cristaefolium extract as recited in claim 1, wherein the hot extraction process comprises:
freeze-drying an algal body of the Sargassum Cristaefolium and grinding the algal body into powder;
heating the powder in a solvent to obtain an extraction fluid; and
collecting a supernatant fraction after centrifuging the extraction fluid, then freeze-drying the supernatant fraction.

3. The use of Sargassum Cristaefolium extract as recited in claim 2, wherein the solvent is distilled water.

4. The use of Sargassum Cristaefolium extract as recited in claim 2, wherein heating the powder is to heat the powder in the solvent at a temperature of 93° C. to 97° C. for 4 hours to 6 hours.

5. The use of Sargassum Cristaefolium extract as recited in claim 2, wherein the extraction fluid is centrifuged at a speed of 2220 rpm.

6. The use of Sargassum Cristaefolium extract as recited in claim 1, wherein the blood lipid regulation function comprises lowering a total cholesterol (TC) concentration in blood.

7. The use of Sargassum Cristaefolium extract as recited in claim 1, wherein the blood lipid regulation function comprises lowering a triglyceride (TG) concentration in blood.

8. The use of Sargassum Cristaefolium extract as recited in claim 1, wherein the blood lipid regulation function comprises enhancing a high-density lipoprotein cholesterol (HDL-C) concentration in blood.

9. The use of Sargassum Cristaefolium extract as recited in claim 1, wherein the blood lipid regulation function comprises lowering a low-density lipoprotein cholesterol (LDL-C) concentration in blood.

10. The use of Sargassum Cristaefolium extract as recited in claim 1, wherein the blood lipid regulation function comprises enhancing an antioxidant activity.
